(19) United States
(12) Patent Application Publication
(10) Pub. No.: US 2022/0287598 A1
(43) Pub. Date: Sep. 15, 2022

(54) BODY MONITORING SYSTEM COMPRISING A MICRONEEDLE

(71) Applicant: PKvitality, Paris (FR)

(72) Inventor: Luc PIERART, VILLEJUIF (FR)

(73) Assignee: PKvitality, Paris (FR)

(21) Appl. No.: 17/633,396

(22) PCT Filed: Aug. 7, 2020
(86) PCT No.: PCT/EP2020/072312

§ 371 Date: Feb. 7, 2022

(30) Foreign Application Priority Data
Aug. 8, 2019 (EP) 19290067.8

(51) Int. Cl. A61B 5/145; A61M 37/00

(52) U.S. Cl. CPC A61B 5/14514; A61M 37/0015; A61M 2037/0053

(57) ABSTRACT

A sensor for a body monitoring system having at least one substrate intended to come into contact with the skin and at least one microneedle, the microneedle being fixedly mounted on the substrate. The substrate has an open channel that surrounds the base of the microneedle, the microneedle having a central axis of symmetry, and the channel having a minimum height in the direction of the central axis that is greater than 20 μm and having a minimum width that is greater than 200 μm in a radial direction to the central axis. The channel forms a cavity in which the microneedle is housed.

### FIELD OF THE INVENTION

The present invention relates to a device for measuring a body analyte. More precisely, it relates to a device for body monitoring by an analysis of bodily fluid, typically interstitial fluid.

### STATE OF THE ART

Certain pathologies, such as diabetes, require daily monitoring of the biochemical parameters of the human body, in particular concentrations in certain compounds (blood glucose, in the example of glucose).

For this, it is common to prick a site on the skin to release a drop of blood and analyze this drop either in a reactive manner (by means of a strip, for example) or electronically (by means of an analytical sensor, for example), so as to estimate the target parameter(s).

Currently, advanced and much less invasive systems are known which simply analyze interstitial fluid, i.e., the fluid that fills the space between blood capillaries and cells. It actually has an ionic composition close to that of blood plasma.

These advanced systems thus make it possible to monitor the desired biochemical parameters transcutaneously, without needing to regularly pierce the skin and draw a sample.

In particular, a device worn on the wrist has been proposed, called GlucoWatch, implementing a phenomenon called iontophoresis (or ionophoresis) in which an electronic field makes it possible to draw the interstitial fluid through the skin to a sensor in the wall of the device. However, this concept was quickly abandoned because only 6% of patients could bear the pain of electrical extraction. In addition, the measurement results were not very reliable.

Alternatively, transcutaneous sensors have been proposed in the form of an adhesive patch placing a “micro-needle sensor” just under the skin, so as to put the sensor in continuous fluid communication with the interstitial fluid, for continuous monitoring. Some of these patch-type transcutaneous sensors comprise wireless communication means making it possible to send measurements of the interstitial liquid to a mobile terminal, for storage and/or processing of the measurements (checking thresholds and variations, producing statistics, triggering alerts if necessary, etc.). Examples include the sugarBEAT™ or FreeStyle Libre systems.

Many proposed systems comprise a microneedle network. However, it has been shown that prolonged and immobile wear of a microneedle network in contact with the skin leads to irritation and promotes bacterial growth.

### DISCLOSURE OF THE INVENTION

The main objective of the invention is to remedy the problems thus posed.

In particular, the present invention aims to propose a sensor comprising a microneedle network that limits irritation and/or bacterial growth when worn by a user.

This goal is achieved in the context of the present invention by means of a sensor for a body monitoring system comprising at least one substrate intended to come into contact with the skin and at least one microneedle, the microneedle being mounted fixedly on the substrate, characterized by the fact that the substrate comprises an open channel that surrounds the base of the microneedle, the microneedle having a central axis of symmetry and the channel having a minimum height in the direction of the central axis greater than 20 μm, and having a minimum width greater than 200 μm in a radial direction to the central axis, the channel forming a cavity in which the microneedle is housed.

The invention is advantageously supplemented by the following characteristics, taken individually or in any one of the technically-possible combinations thereof:
the channel has a minimum height in the direction of the central axis greater than 100 μm and has a minimum width greater than 1000 μm in a radial direction to the central axis, the channel forming a cavity in which the microneedle is housed,
the substrate comprises at least one open passage, and preferentially at least two open passages, ensuring a connection between the channel and the outside so as to renew the air surrounding the base of one or more microneedles 20,
the substrate has the general shape of a ring having a central orifice and the passage connects the channel of each needle to the central orifice,
the passage has a minimum height in the direction of the central axis greater than 100 μm and has a minimum width greater than 100 μm in a radial direction to the central axis,
the support substrate comprises at least one opening connecting at least two channels, each channel surrounding the base of at least one microneedle,
the substrate comprises at least one open passage common to at most two hundred microneedles and preferentially at most eight microneedles,
the sensor comprises at least one passage connecting the channel to the outside and comprises a barrier of semipermeable material, i.e., impermeable to water and permeable to air,
the sensor comprises one passage connecting the channel to the outside, said passage having a section designed to prevent the entrance of water by capillary repulsion.

Another aspect of the invention is a body monitoring system comprising a sensor according to the invention and also comprising a module configured to exploit an electrical signal issued by the sensor And provide information representative of an analyte.

Preferably, this system also comprises at least one opening connecting the at least one channel to the outside of the system.

Another aspect of the invention is a body monitoring method comprising a step in which a sensor according to the invention is used.

### DESCRIPTION OF THE FIGURES

Other characteristics, objectives and advantages of the present invention will appear upon reading the detailed description that follows in reference to the drawings given by way of non-limiting examples and in which:

FIG. 1 is a perspective view of a sensor comprising four microneedles,

FIG. 2 is a perspective view along a different angle of observation than FIG. 1 of such a sensor,

FIG. 3 represents a rear face of such a sensor,

FIG. 4 represents a side view of such a sensor,

FIG. 5 represents a tip side view of the sensor,

FIG. 6 represents a lateral view of a microneedle according to one embodiment of the invention,

FIG. 7 is a schematic perspective view of a body monitoring system incorporating a sensor according to the present invention, 15

FIG. 8 represents a view of a capsule designed to bear a plurality of sensors according to the invention,

FIG. 9 represents an enlarged scale view of a part of this capsule,

FIG. 10 represents an enlarged scale view of a part of a capsule according to a different embodiment of the invention,

FIG. 11 schematically illustrates a section of a microneedle mounted fixedly to a substrate forming an air channel which surrounds the base of the microneedle.

### DEFINITION

“Electrode” means a conductive device for sensing variations in electrical potential in a living organism. An electrode comprises a terminal having a connection end and at least one detection end by which an electrical potential or electrical current is transmitted, each detection end being borne by one microneedle. The electrode can thus comprise a single detection end. it can also comprise a plurality of detection ends. In this case, it will be noted that the electrode remains unique, even if several detection ends are intended to penetrate into the body of a living organism.

### DETAILED DESCRIPTION OF THE INVENTION

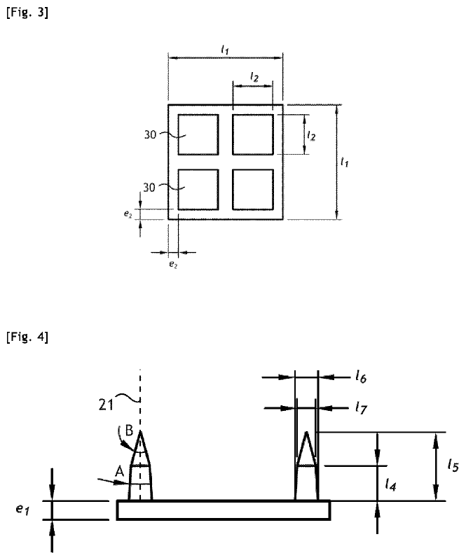

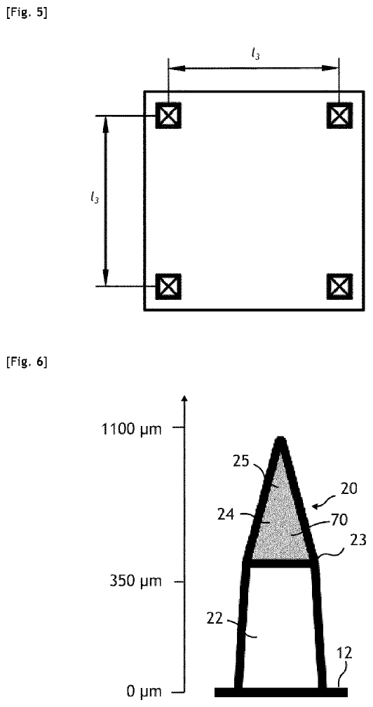

A particular and non-limiting embodiment of a sensor according to the present invention will be described such as illustrated in FIGS. 1 to 6 attached.

### Microneedle(s)

In reference to FIGS. 1 to 6, a sensor comprises a support plate 10 provided with four microneedles 20. The outline of the plate can be subject to numerous variations of embodiment. According to the representation given in FIGS. 1 to 5, plate 10 has a square outline. The four microneedles are respectively located near the corners of the plate 10. The microneedles 20 extend perpendicularly to the base plane of plate 10. In other words, the central axis 21 of each microneedle 20 extends perpendicularly to the surface of the base of plate 10.

In reference to FIGS. 1 to 3, the face of the plate 10 opposite the microneedles 20 comprises four electrically conductive pads 30, each pad 30 being electrically connected to the active part 25 of each microneedle 20. The electrically conductive pads 30 allow electrical continuity of an electrode when they are electrically connected to a base, for example, a working or a counter-electrode, in the microneedle 20, potentially in a separate manner so that each working electrode is independent of the other working electrodes. In practice, these pads 30 can be integral with the microneedles 20 or electrically connected to the microneedles 20 by any appropriate means through or around the plate 10. The support plate 10 can be made of any electrically-appropriate material, for example electrically insulating or conductive. Likewise, microneedles 20 can be formed of any appropriate material. They are able to carry an electrical signal captured by the active surface 25. Preferentially, the microneedles 20 can be formed based on polycarbonate or silicon. Microneedle(s) 20 are preferentially solid, i.e., do not have cavities. Thus, the manufacture of microneedles 20 suited to measuring the analyte is facilitated, while allowing electrochemical measurement of an analyte. The microneedle can predominantly comprise silicon. In the case of silicon, the microneedle has an external, nonconducting SiO₂ protective layer, formed by oxidation of silicon on the surface. Thus the microneedle does not comprise additional coating beyond the SiO₂ layer.

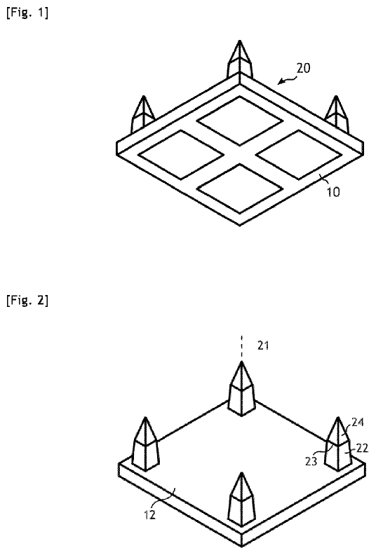

In reference to FIG. 2 and FIG. 6, each microneedle 20 comprises a base shaft 22 and a pointed tip 24. Shaft 22 can preferentially become thinner toward the pointed tip 24 of the microneedle 20. The pointed tip 24 has a slope greater than that of the shaft 22, i.e., it forms an angle B with the central axis 21 greater than the angle A formed between the base shaft 22 and the central axis 21. Microneedle 20 has a slope break or transition 23 between the base shaft 22 and the pointed tip. The slope transition 23 can be embodied by an edge.

The base shaft 22 and the pointed tip 24 can have a square section. In this case, the base shaft 22 is quadrangular and the tip 24 is pyramidal. The entirety of microneedle 20 can preferentially have the shape of an obelisk.

However, as a variant, the microneedle 20 can have a circular section. In this case, the base shaft 22 has the shape of a circular truncated cone of revolution and the tip 24 is formed of a conical tip of revolution.

Preferably, the pointed tip 24 extends exclusively at a distance comprised between 350 μm and 1100 μm from the base of the base shaft 22 of the microneedle, i.e., from the face 12 of the support plate 10, and preferably between 600 μm and 1000 μm from the base of the microneedle and this surface 12 of the support plate 10. The term “extends exclusively at a distance comprised between 350 μm and 1100 μm” means that the part of the pointed tip 24 closest to the base of the base shaft is arranged at a distance greater than 350 μm from the base of the base shaft 22, and that the most distant part from the base of the base shaft 22 is arranged at a distance of less than 1100 μm.

Moreover, tests have shown that the area of active detection part 25 must be comprised between 0.04 and 0.9 mm². Consequently, when the measurement is performed with a single microneedle 20, the active part 25 of this microneedle 20 is comprised between 0.04 and 0.9 mm². When the measurement is performed with several microneedles 20, the above-mentioned area of the active part, comprised between 0.04 and 0.9 mm², means the total active surface of the microneedles considered.

The skilled person will understand that the obelisk shape of the microneedles 20, or the shape of a similar circular body of revolution but having a break in slope between the base shaft 22 and the pointed tip 24, makes it possible to solve a problem posed by known microneedles 20 of the state of the art, i.e., minimizing the diameter of penetration into the skin while maximizing the surface area of the active part 25 present in the part of the skin comprised between the epidermis and the nerves.

The microneedles 20 according to the present invention can be made using any appropriate micromanufacturing method.

The active part 25 comprises an electrically conductive face, preferentially covered with a coating that is subject to diverse variations depending on the type of measurement sought and the type of analyte to be measured. For measuring blood glucose, the active part 25 is provided with a coating suited to implement an enzymatic reaction with glucose. The active part 25 can also not comprise coating specific to a predetermined analyte, for example in the case of the active part 25 of a counter electrode or a reference electrode.

Moreover, according to the particular embodiment represented in FIGS. 1 to 5, and by way of non-limiting example:
the height l₄ of base shaft 22 is around 380 μm,
the total height l₅ of each microneedle 20 is around 750 μm,
the width of the base l₆ of each microneedle 20 is around 0.25 mm,
the width l₇ of the microneedle at the slope transition 23 is around 0.2 mm,
the angle A of convergence of the base shaft 22 relative to the central axis 21 is around 7°,
the angle B of the pointed tip 24 relative to the central axis 21 is around 30°.

### Microneedle Network

The microneedle(s) according to the invention make it possible to reduce the number of microneedles 20 of a working electrode 70. Preferentially, a working electrode comprises between one and seven, especially between one and five and preferentially between one and three active parts 25 each covering at least one part of the surface of the pointed tip 24 of a different microneedle 20. Known systems of the state of the art do not allow using so few microneedles.

Since the invention makes it possible to drastically reduce the number of microneedles 20 necessary for the measurement relative to known systems of the state of the art, it is possible, for a given sensor surface, to minimize the density of the microneedles. Each adjacent pair of microneedles 20 is preferentially separated for a distance between the points of pointed tips 24 of at least 1 mm and preferentially at least 1.5 mm, or even, as appropriate, at least 1.8 mm. The effect of this is to prevent a homogeneous deformation of the skin when the network of microneedles 20 is brought into contact with the skin, known in other technical fields by the name fakir effect, and, on the contrary, to promote a localized deformation of the skin around each of the microneedles. Thus, the pain caused by the penetration of the needles into the skin can be significantly reduced, even eliminated and the penetration is done very naturally, the needles having in fact become mechanically independent.

Moreover, according to the particular embodiment represented in FIGS. 1 to 5, and by way of non-limiting example, the center distance l₃ between each pair of microneedles 20 is of the order of 1.5 mm.

### Electrodes

The sensor is preferentially designed to measure the presence or concentration of an analyte by electrochemistry. A sensor can comprise a working electrode 70, designed to assess the presence of an analyte in the user's body. The working electrode 70 comprises at least one first end electrically connected to a module configured to exploit the electrical signal from the working electrode 70, and at least one second end formed by the active part 25. It can also comprise a plurality of second ends. The active part 25 of the microneedle 20 covers at least a part of the surface of the pointed tip 24 and preferentially all of the surface of the pointed tip 24. To this end, the active part 25, at the pointed tip 24, is coated with any appropriate coating for the desired measurement, typically a coating designed to detect blood glucose by electrochemistry.

The sensor can comprise a counter-electrode. The counter-electrode can comprise a first end intended to be electrically connected to a module configured to exploit an electrical signal, and at least one other end that can exploit an electrical signal in the user's body. The other end of the counter electrode can cover a microneedle of the counter electrode, for example a microneedle according to the invention. However, the counter-electrode does not have the same active surface prerequisites as the working electrode. Thus, the pointed tip of the counter electrode can extend exclusively to a distance comprised between 100 μm and 1100 μm from the base shaft of the microneedle. Alternatively, the other end of the counter-electrode can cover the entire surface of the counter-electrode microneedle.

In the case of a sensor according to an embodiment of the invention comprising several working electrodes, each working electrode can be designed to detect the same analyte as another working electrode or be designed to detect an analyte different than another working electrode.

In the case where several working electrodes are designed to detect the same analyte, each working electrode can comprise an active part 25 comprising the same type of coating. Thus, it is possible to implement independent measurements for the same analyte, and thus to obtain a better measurement precision for the analyte. Each working electrode can also comprise different active parts 25, comprising different coatings, but designed to detect the same analyte. The concentration of the analyte can thus be detected with more precision than by using a single coating for active part 25.

Each electrode can also be designed to detect different analytes. Thus it is possible to monitor several diseases with the same monitoring system.

### Support Plate

As seen previously, the microneedle(s) 20 can be arranged on a support plate 10. In reference to FIGS. 1 to 4, the thickness e₁ of the support plate is advantageously comprised between 0.1 mm and 1 mm, and preferentially around 0.2 mm. The dimensions of the microneedles 20 can be subject to numerous variants of embodiment. It is the same for the support plate 10. By way of non-limiting example, in reference to the particular embodiment represented in FIGS. 1 to 5, the support plate 10 has sides with a width l₁ less than 10 mm, advantageously less than 3 mm, for example around 2.3 mm. Electrically conductive pads 30 are pads, for example, square, having a side l₂ of around 0.8 mm. These pads 30 can be located at a distance e₂ of around 0.2 mm from the edges of the support plate.

The support plates 10 can themselves be subject to different variants of embodiment. Some support plates 10 can be designed to support four microneedles, for example, while other support plates 10 can be designed to support only two microneedles 20.

### Measurement System

The present invention makes it possible to independently measure blood glucose using a plurality of working electrodes 70. This has an undeniable advantage relative to the state of the art according to which such an independent measurement by means of a working electrode comprising a single microneedle was not possible because the measurement signal was too noisy by using a single microneedle. Thus, the sensor preferentially comprises several working electrodes 70, since the measurement system is designed to individually measure the electric potential of each of the electrodes 70.

The measurement of the potential of each of the working electrodes 70 can be multiplexed. In the context of the present invention, means have advantageously been proposed making it possible to reject the minimum or maximum measurement values of the potential associated with a set of measurements, which was impossible by using sensors of the prior art.

The sensor according to the present invention can be implemented in different types of body monitoring systems.

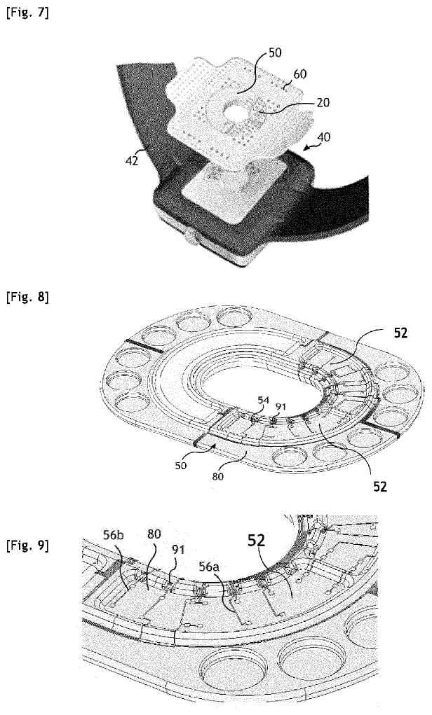

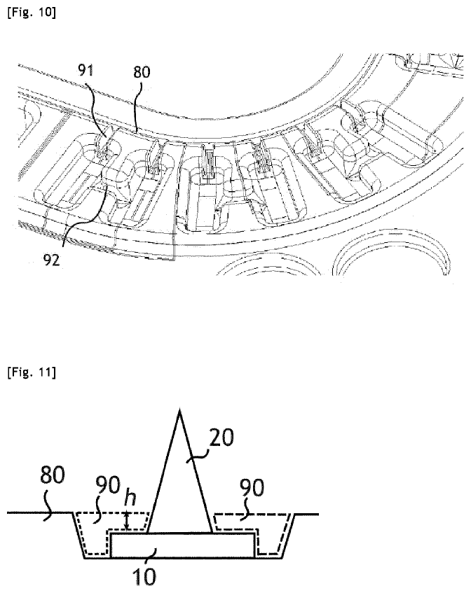

Preferably, the sensor according to the invention is implemented in a body monitoring system of the type illustrated in FIGS. 7 to 10 attached.

Such a system comprises a casing 40 in the form of a watch casing comprising a bracelet 42 designed to surround the wrist of an individual. The casing 40 houses a module configured to exploit the electrical signal delivered from each microneedle 20 and provide information representative of a physical quantity of the fluid, typically a blood glucose level.

As stated previously, the body monitoring system preferentially used according to the invention comprises a capsule 50 comprising at least one sensor of the above-mentioned type, and preferably a plurality of sensors such as will be described in more detail below.

The body monitoring system according to the invention also comprises a patch 60 which is connected to the capsule 50, the patch 60 being itself provided with an adhesive to allow affixing the patch and capsule 50 assembly on the skin of an individual.

As is shown in FIG. 8 and in part in FIG. 9, the capsule 50 preferably has the general shape of a ring comprising a plurality of hollow recesses 52 designed to each respectively receive the support plate 10 of an above-mentioned sensor.

In reference to FIGS. 7 and 8, the capsule 50 can comprise electrically-conductive pads 54 designed to be positioned opposite the electrically-conductive pads 30 provided on the support plate 10, to ensure an electrical connection between the microneedles 20 and the module provided in the casing 40 to exploit the electrical signal thus sampled. The pads 54 are themselves interconnected with the same above-mentioned module by electrically-conductive tracks 56a.

As is seen on examining FIG. 9, some of the pads 54 can be individually connected to the above-mentioned treatment module by the respective tracks 56a while other pads 54 can be connected to the processing module by common tracks 56b.

### Limitation of Irritation

In reference to FIGS. 8, 9, 10 and 11, the sensor comprises a substrate on which the microneedles are fixedly mounted. Substrate means, for example, the assembly of a support plate 10 and a support base 80, or the surface of a capsule 50. The substrate, or at least a part of the substrate, is intended to come into contact with the user's skin. The substrate comprises an open channel 90, which surrounds the base of the microneedle(s) 20. Preferentially, one wall of one or more microneedle(s) forms a wall of the channel 90. “Open” channel means that one of the walls of the channel 90 or a part of the walls of the channel 90 can be missing, the channel 90 being designed so that the user's skin, when it is in contact with the substrate, comes to form the missing wall of the channel 90. Thus, when the skin is in contact with the substrate, an air channel 90 surrounds microneedle(s) 20. This maintains the opening of the skin caused by the penetration of the microneedle in contact with the air. In this way, irritation is prevented.

The skin has a suppleness that allows it to adapt to different types of nonplanar shapes. The channel has a minimum height h in the direction of the central axis 21 of a microneedle greater than 20 μm. Thus, the skin does not sink into the channel 90 for high pressures exerted by the skin on the substrate, and the channel 90 cannot be blocked by the skin. Advantageously, the minimum height h of the channel is greater than 100 μm.

The channel preferentially has a minimum width in a direction radial to the central axis 21 greater than 200 μm and preferentially than 1000 μm. This makes it possible to retain a sufficient reservoir of air for the skin to heal around the microneedle 20.

Channel 90 can be formed by many geometries. Preferentially, and in reference to FIG. 11, the substrate can have a cavity in which the microneedle 20 is housed, forming the open channel 90 around the base of the microneedle 20.

The channel 90 can be connected to the outside of the sensor and/or system by a passage 91. The passage 91 can be defined by the substrate, like the channel 90. The passage 91 can be an open passage 91, i.e., one of the walls of the passage 91 or a part of the walls of the passage 91 can be missing, the passage 91 being designed so that the user's skin, when it is in contact with the substrate, comes to form the missing wall of the passage 91. Thus, the air surrounding the base of one or more microneedles 20 can be renewed via the passage 91, which makes it possible to regulate the humidity level in the channel 90 and prevent skin irritation. The passage 91 can have a minimum height in the direction of the central axis 21 greater than 100 μm and has a minimum width greater than 100 μm in a transverse direction to the central axis 21. Thus, it is possible to ensure a sufficient airflow in the direction of channel 90 or in the direction of the outside, so as to prevent skin irritation. “Connected” means that the passage 91 has a fluid outlet that also defines a fluid inlet for the channel 90, the passage 91 and channel 90 being connected directly.

The substrate can preferentially have the general shape of a ring having a central orifice. The passage 91 can connect the channel 90 to the central orifice. Thus, it is possible to protect the intrusion of elements harmful to the healing of the skin directly from the outside of the sensor or device.

The passage has a minimum width of at least 100 μm and preferentially of at least 300 μm in a direction transverse to the central axis 21. Thus, the renewal of air coming from outside the sensor can be ensured without risk of blocking the passage 91.

The sensor can also comprise at least one opening 92 to connect the at least two channels 90. Thus, if one of the channels 90 is not connected by a passage 91, the air surrounding the microneedle(s) 20 can still be renewed via opening 92. A passage 91 can thus be preferentially common to at most two hundred microneedles, advantageously at most sixteen microneedles and preferentially at most eight microneedles. Beyond this, supplying air can be more difficult.

The passage 91 can preferentially comprise a barrier of semipermeable material, i.e., impermeable to water and permeable to air. Thus, if the sensor and/or the system is accidently contacted with water, the introduction of water around the needles can be prevented. Alternatively, it is possible to define a width of the passage(s) 91 small enough to prevent the introduction of water by capillary repulsion. The width of the passage 91 can thus be less than 500 μm and preferentially less than 400 μm.

### Implementation of a Measurement of a Body Analyte

As indicated previously, the present invention also concerns a method for body monitoring using a sensor comprising a microneedle of the above-mentioned type.

The monitoring method comprises a step of measuring a body analyte using a microneedle 20 according to an embodiment of the invention. Due to the characteristics of the microneedle 20, the sensor can comprise a plurality of working electrodes. The measurement can be implemented by polarizing the working electrode(s) and the counter-electrode(s) at an appropriate electrical potential to cause a redox reaction involving the analyte to be measured.

The measurement step can preferentially be implemented using at least two different working electrodes. The measurement step can, for example, be implemented independently, successively on each of the working electrodes 70 or at the same time on each of the working electrodes 70. Thus, the concentration of the electrolyte can be analyzed more precisely than with a system comprising, for example, a single working electrode having several ends in the form of microneedles.

Another aspect of the invention is a method for measuring a body analyte comprising a step of penetration of the microneedles of a sensor according to the invention into a user's skin. Thus, the needles of the sensor can be introduced into the skin without an applicator, by the spacing of the microneedles 20. A small force in comparison to the force procured by an applicator can be used for the penetration of the microneedles. Preferentially, a force less than 50 Newtons, and preferentially less than 30 Newtons, can be used for the penetration of the microneedles 20. Thus, the penetration of the microneedles can be implemented by hand, or preferentially with the system's means for mechanical attachment, for example a bracelet.

1. Sensor for a body monitoring system comprising: at least one substrate intended to come into contact with the skin and at least one microneedle, the microneedle being mounted fixedly on the substrate and comprises a base, wherein the substrate comprises an open channel surrounding the base of the microneedle, the microneedle having a central axis of symmetry and the open channel having a minimum height in a direction of the central axis of symmetry greater than 20 μm, and having a minimum width greater than 200 μm in a radial direction to the central axis, the channel forming a cavity in which the microneedle is housed, the substrate comprising at least one open passage defined by the substrate and ensuring a connection between the channel and the outside so as to renew the air surrounding the base of one or more microneedles 20.

2. Sensor according to claim 1, wherein the channel has a minimum height (h) in the direction of the central axis greater than 100 μm and has a minimum width greater than 1000 μm in a radial direction to the central axis of symmetry, the channel forming a cavity in which the microneedle is housed.

3. Sensor according to claim 1, wherein the substrate has a general shape of a ring having a central orifice and the passage connects the open channel of each microneedle to the central orifice.

4. Sensor according to claim 3, wherein the passage can have a minimum height in the direction of the central axis greater than 100 μm and has a minimum width greater than 100 μm in a transverse direction to the central axis.

5. Sensor according to one claim 1, wherein the support substrate comprises one opening connecting at least two channels, each channel surrounding the base of at least one microneedle.

6. Sensor according to claim 1, wherein the substrate comprises at least one open passage common to at most two hundred microneedles, advantageously at most sixteen microneedles and preferentially at most eight microneedles.

7. Sensor according to claim 1, comprising at least one passage connecting the channel to the outside and comprising a barrier of semipermeable material, i.e., impermeable to water and permeable to air.

8. Sensor according to claim 1, comprising at least one passage connecting the channel to the outside, said passage having a section designed to prevent the entrance of water by capillary repulsion.

9. Body monitoring system comprising a sensor according to claim 1, comprising a module configured to exploit an electrical signal issued by the sensor and provide information representative of an analyte.

10. System according to claim 9, comprising at least one opening connecting the at least one channel (90) to the outside of the system.

11. Method for body monitoring comprising a step in which a sensor for the body monitoring system according to claim 1 used.
